(19) United States
(12) Patent Application Publication
(10) Pub. No.: US 2010/0197841 A1
(43) Pub. Date: Aug. 5, 2010

(54) HYDROXY ACID AMINE SALT, METHOD FOR PRODUCING THE SAME, AND RUBBER COMPOSITION CONTAINING THE SAME

(75) Inventors: Takashi Matsuda, Kanagawa-ken (JP); Wonmun Choi, Kanagawa-ken (JP)

Correspondence Address: THORPE NORTH & WESTERN, LLP., P.O. Box 1219, SANDY, UT, 84091-1219, US

(73) Assignee: The Yokohama Rubber Co., Ltd.

(21) Appl. No.: 12/669,864

(22) PCT Filed: Jul. 18, 2008
(86) PCT No.: PCT/JP08/63471

§ 371 Date: Apr. 14, 2010

(30) Foreign Application Priority Data
Jul. 20, 2007 (JP) 2007-189810

(51) Int. Cl. C08K 5/17; C07C 229/48; C07C 227/00

(52) U.S. Cl. 524/186; 562/500

(57) ABSTRACT

Disclosed is a hydroxy acid amine salt represented by the general formula (I) below. Also disclosed are a method for producing such a hydroxy acid amine salt, and a rubber composition containing such a hydroxy acid amine salt. (In the formula, R¹, R², R³, R⁴, k, l, m and n are as defined in the description.)

### TECHNICAL FIELD

The present invention relates to a new hydroxy acid amine salt, a method for producing the same, and a rubber composition containing the hydroxy acid amine salt. In particular, the present invention relates to a hydroxy acid amine salt useful for improving the vulcanization characteristics and viscoelastic characteristics of rubber including silica, a method for producing the same, and a rubber composition including such hydroxy acid amine salt.

### BACKGROUND ART

When producing rubber compositions by adding various compounding agents such as reinforcing fillers to a rubber component, in addition to showing good viscoelastic properties after vulcanization, securing good vulcanization characteristics for production improvement is desired. Thus, many compounds have been suggested as compounding agents for rubber. For example, Japanese Patent Application Publication No. 2003-138077A suggests adding a specific carboxylic acid amine salt having vulcanization accelerating properties to a halogen-based saturated rubber. Although a certain effect is achieved, further improvements in vulcanization characteristics and viscoelastic properties are desired.

### DISCLOSURE OF THE INVENTION

Accordingly, objects of the present invention are to provide a hydroxy acid amine salt that, together with increasing vulcanization efficiency of rubber compositions, improves the viscoelastic characteristics of the obtained rubber compositions after vulcanization, and to provide a method for producing the same. Rubber compositions having superior vulcanization characteristics and viscoelastic characteristics are provided by adding the hydroxy acid amine salt of the present invention to the rubber compositions.

### BEST MODE FOR CARRYING OUT THE INVENTION

The present inventors, as a result of diligent study considering the above-mentioned problems, have found that a hydroxy acid amine salt represented by the following general formula (I):

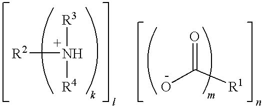

(wherein R¹ represents a saturated or unsaturated organic group of 1 to 12 carbons having 1 or more hydroxyl groups;

R², R³, and R⁴ each independently represent a hydrogen atom, a saturated or unsaturated chain hydrocarbon group of 1 to 12 carbons, a saturated or unsaturated alicyclic hydrocarbon group of 3 to 12 carbons, an aromatic hydrocarbon group of 6 to 18 carbons, or a heterocyclic group of 5 to 18 ring atoms and 2 or more carbons; or at least two of R², R³, and R⁴ join together with nitrogen atoms to which they are bonded to form a heterocyclic group of 5 to 18 ring atoms and 2 or more carbons; and k, l, and n are each integers of 1 or more, and m is an integer of 2 or more, with a proviso that k, l, m, and n satisfy a relational expression such that k×l=m×n=2 or more)

increases vulcanization efficiency of rubber compositions and, furthermore, improves the viscoelastic characteristics of rubber compositions after vulcanization, thus completing the present invention.

That is, the hydroxy acid amine salt represented by the above-mentioned general formula (I) is provided in a first aspect of the present invention.

A method for producing the hydroxy acid amine salt represented by the above-mentioned general formula (I) is provided in a second aspect of the present invention.

A rubber composition containing the hydroxy acid amine salt represented by the above-mentioned general formula (I) is provided in a third aspect of the present invention.

The hydroxy acid amine salt represented by the above-mentioned general formula (I) of the first aspect of the present invention has one or more hydroxy groups in the carboxylic acid anion moiety. As specific examples of R¹ in the above-mentioned general formula (I)

(1) hydroxy-substituted derivatives of saturated or unsaturated chain hydrocarbon groups of 1 to 12 carbons, for example, hydroxy-substituted derivatives of alkylene, alkenylene, and alkynylene groups such as hydroxy-substituted derivatives of a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, a dodecamethylene group, a propylene group, a vinylene group, a propenylene group, a —CH═CH—(CH₂)₈— group, an ethenylene group, a —CH≡CH—CH₂— group, a 1,2,3-triylpropane group and the like;

(2) hydroxy-substituted derivatives of saturated or unsaturated hetero atom-containing chain hydrocarbon groups of 1 to 12 carbons such as —CH₂—O—CH₂—, —CH₂—S—CH₂—, (—CH₂—)₂N—(CH₂)₂—N(—CH₂—)₂, (—CH₂—)₂N—(CH₂)₂—N(—CH₂—)—(CH₂)₂—N(—CH₂—)₂, (—CH₂—)₂N—(CH₂)₂—N(—CH₂—)—(CH₂)₂—N(—CH₂—)—(CH₂)₂—N(—CH₂—)₂, —CH₂CH₂CH—N(CH₂)₂—, and the like;

(3) hydroxy-substituted derivatives of saturated or unsaturated alicyclic hydrocarbon groups of 3 to 12 carbons such as hydroxy-substituted derivatives of a cyclopropylidene group, a cyclopropylene group, a cyclobutylene group, a cyclopentylene group, a cyclohexylene group, a cyclohexenylene group, a cyclooctylene group, 1-methyl-4-cyclohexenylene group, a norbornylene group, and the like;

(4) hydroxy-substituted derivatives of aromatic hydrocarbon groups of 6 to 12 carbons, for example, hydroxy-substituted derivatives of aryl, aryl alkyl, aryl alkenyl, alkyl aryl, or alkenyl aryl groups such as hydroxy-substituted derivatives of an o-phenylene group, an m-phenylene group, a p-phenylene group, a 4-methyl-m-phenylene group, a cyclooctatetraenylene group, a 1,2,-naphthalenylene group, a 1,3-naphthalenylene group, and 1,8-naphthalenylene group, a naphthalenetriyl group, a biphenylenediyl group, a biphenyldiyl group, a C₆H₅—CH₂—CH— group, a —CH₂—C₆H₄—CH₂— group, and the like; and

(5) hydroxy-substituted derivatives of heterocyclic groups of 5 to 18 ring atoms and 2 or more carbons, for example, hydroxy-substituted derivatives of organic groups such a furandiyl group, a thiophenediyl group, a pyrrolediyl group, an oxazolediyl group, an isooxazolediyl group, a thiazolediyl group, and isothiazolediyl group, an imidazolediyl group, a pyrazolediyl group, a triazolediyl group, a pyridinediyl group, a pyrimidinediyl group, a pyridazinediyl group, a piperidinediyl group, a piperazinediyl group, a morpholinediyl group, an indolediyl group, an isoindolediyl group, a benzofurandiyl group, a benzothiophenediyl group, a quinolinediyl group, an acridinediyl group, and the like may be mentioned.

Preferably, R¹ is a saturated or unsaturated chain hydrocarbon group of 1 to 12 carbons having one or more hydroxyl groups, or is an aromatic hydrocarbon group of 6 to 12 carbons having one or more hydroxyl groups. More preferably, R¹ is a hydrocarbon group of 1 to 8 carbon atoms having one or more hydroxyl groups, for example, a hydroxymethylene group (—CH(OH)—), a monohydroxyethylene group (—CH₂CH(OH)—), a 1,2-dihydroxyethylene group (—CH(OH)—CH(OH)—), a 1-hydroxy-1,2,3-triylpropane group (—CH(OH)—CH₂—CH₂—), a 2-hydroxy-1,2,3-triylpropane group (—CH₂—CH(OH)—CH₂—), a hydroxyphenylene group (—C₆H₃(OH)—), or the like. Even more preferably, R¹ is a monohydroxyethylene group (—CH₂CH(OH)—) or a 1,2-dihydroxyethylene group (—CH(OH)—CH(OH)—). R¹ is particularly preferably a monohydroxyethylene group (—CH₂CH(OH)—) or a 1,2-dihydroxyethylene group (—CH(OH)—CH(OH)—).

As specific examples of R², R³, and R⁴ in the above-mentioned general formula (I), hydrogen atoms, saturated or unsaturated chain hydrocarbon groups of 1 to 12 carbons, for example, alkyl, alkenyl, and alkynyl groups such as methyl groups, ethyl groups, propyl groups, isopropyl groups, butyl groups, isobutyl groups, sec-butyl groups, tert-butyl groups, pentyl groups, isopentyl groups, neopentyl groups, tert-pentyl groups, hexyl groups, isohexyl groups, 2-ethylhexyl groups, heptyl groups, octyl groups, nonyl groups, decyl groups, undecyl groups, dodecyl groups, vinyl groups, allyl groups, isopropenyl groups, —CH═CH—(CH₂)₈— groups, ethynyl groups, and the like; saturated or unsaturated alicyclic hydrocarbon groups of 3 to 12 carbons such as cyclopropyl groups, cyclopropenyl groups, cyclobutyl groups, cyclopentyl groups, cyclohexyl groups, cyclohexenyl groups, cycloheptyl groups, cyclooctyl groups, adamantanyl groups, and the like; along with aromatic hydrocarbon groups of 6 to 18 carbons, for example, aryl groups such as phenyl groups, naphthyl groups, biphenylenyl groups, fluorenyl groups, anthryl groups, and the like, aryl alkyl groups such as benzyl groups, phenylethyl groups, and the like, aryl alkenyl groups such as styryl groups, cinnamyl groups, and the like, alkyl aryl groups such o-, m-, or p-methylphenyl groups, 2,3-, 2,4-, 2,5-, 2,6-, 3,4-, or 3,5-dimethylphenyl groups, 2,4,5- or 2,4,6-trimethylphenyl groups, 2,3,4,5-, 2,3,4,6-, or 2,3,5,6-tetramethylphenyl groups, 3-, 4-, 5-, or 6-ethyl-2-hexylphenyl groups, 2-, 4-, or 5-ethyl-3-hexylphenyl groups, 2- or 3-ethyl-4-hexylphenyl groups, 2- or 3-ethyl-5-hexylphenyl groups, 2-ethyl-6-hexylphenyl groups, and the like, alkenyl aryls such as styryl groups, alkoxy-substituted derivatives of aryl, aryl alkyl, aryl alkenyl, alkyl aryl, and alkenyl aryl groups, for example, 2-, 3-, or 4-methoxyphenyl groups, 2-, 3-, or 4-ethoxyphenyl groups, 2-, or 4-methoxy-3-methylphenyl groups, 2- or 3-methoxy-4-methylphenyl groups, 2- or 3-methoxy-5-methylphenyl groups, 2- or 3-methoxy-6-methylphenyl groups, 3- or 4-methoxy-2-methylphenyl groups, 2- or 4-methoxy-3-ethylphenyl groups, 2- or 3-methoxy-4-ethylphenyl groups, 2-, 3-, or 4-methoxy-5-ethylphenyl groups, 2-, 3-, or 4-methoxy-6-ethylphenyl groups, 3- or 4-methoxy-2-ethylphenyl groups, 2-methoxy-3,4,5-trimethylphenyl groups, 3-methoxy-2,4,5-trimethylphenyl groups, 4-methoxy-2,3,5-trimethylphenyl groups, and the like; and heterocyclic groups of 5 to 18 ring atoms and 2 or more carbons, for example, furanyl groups, thienyl groups, pyrrolyl groups, oxazolyl groups, isooxazolyl groups, thiazolyl groups, isothiazolyl groups, imidazolyl groups, pyrazolyl groups, triazolyl groups, pyridinyl groups, pyrimidinyl groups, pyridazinyl groups, piperidinyl groups, piperazinyl groups, morpholinyl groups, indolyl groups, isoindolyl groups, benzofuranyl groups, benzothienyl groups, quinolinyl groups, acridinyl groups, 2-(3,4-dihydroxyphenyl)ethylene groups, 2-(3,4-dihydroxyphenyl)-2-hydroxyethyl groups, and the like may be mentioned.

The above-mentioned saturated or unsaturated chain hydrocarbon groups of 1 to 12 carbons can be a straight chain or a branched chain. Also, concerning the above-mentioned saturated or unsaturated alicyclic hydrocarbon groups of 3 to 12 carbons, the aromatic hydrocarbon groups of 6 to 18 carbons, and the heterocyclic groups of 5 to 18 ring atoms and 2 or more carbons, when a substituent is present on the carbon ring or the hetero ring, the above-mentioned number of carbons means the total number of carbons including the number of carbons of the substituent.

At least two among R², R³, and R⁴ may join together with the nitrogen atoms to which they are bonded to form a heterocyclic group of 5 to 18 ring atoms and 2 or more carbons, for example, furanyl groups, thienyl groups, pyrrolyl groups, oxazolyl groups, isooxazolyl groups, thiazolyl groups, isothiazolyl groups, imidazolyl groups, pyrazolyl groups, triazolyl groups, pyridinyl groups, pyrimidinyl groups, pyridazinyl groups, piperidinyl groups, piperazinyl groups, morpholinyl groups, indolyl groups, isoindolyl groups, benzofuranyl groups, benzothienyl groups, quinolinyl groups, acridinyl groups, and the like.

Preferably, R², R³, and R⁴ are each independently selected from a hydrogen atom, a saturated or unsaturated chain hydrocarbon of 4 to 6 carbons, a saturated or unsaturated alicyclic hydrocarbon group of 6 carbons, and an aromatic hydrocarbon group of 6 to 8 carbons. More preferably, R² is a cyclic or chain alkyl group of 2 to 10 carbons, for example, an ethyl group, a propyl group, a butyl group, a hexyl group, a cyclohexyl group, or the like, and R³ and R⁴ are hydrogen atoms or cyclic or chain alkyl groups of 2 to 10 carbons.

In general formula (I), k is preferably an integer of 1 or 2, 1 is preferably an integer of 1 to 3, m is preferably an integer of 2 or 3, and n is preferably an integer of 1 to 3. More preferably, k=1, l=2, m=2, and n=1 or k=1, l=3, m=3, and n=1. However, k, l, m, and n satisfy the relational expression of k×l=m×n=2 or more.

The method for producing the hydroxy acid amine salt represented by the above-mentioned general formula (I) of the second aspect of the present invention is such that the hydroxy acid amine salt represented by the above-mentioned general formula (I) is produced by reacting n moles of a hydroxy acid represented by the following general formula (II):

R¹—(COOH)ₘ (II)

and 1 mole of an amine represented by the following general formula (III):

R²—(NR³R⁴)ₖ (III).

Here, R¹, R², R³, and R⁴ are as previously defined. k, l, and n are each integers of 1 or more and m is an integer of 2 or more, with the proviso that k, l, m, and n satisfy the relational expression k×l=m×n=2 or more.

The hydroxy acid amine salt of the above-mentioned general formula (I) can be synthesized by reacting the hydroxy acid of general formula (II) and the amine of general formula (III) with or without a solvent. When the hydroxy acid amine salt of general formula (I) is synthesized with a solvent, the reaction temperature can be determined considering the volatility and stability of the solvent, hydroxy acid, and amine used, and the solubility of the hydroxy acid and amine in the solvent. When the hydroxy acid amine salt of general formula (I) is synthesized without a solvent, the reaction temperature can be determined considering the mutual solubility between the hydroxy acid and amine, and the hydroxy acid amine salt of general formula (I) can be obtained by reacting these. The amine group included in the amine of the above-mentioned general formula (III) is quaternized by the reaction with the carboxyl group of the hydroxy acid of the above-mentioned general formula (II), and the amine salt of the above-mentioned general formula (I) is formed by this reaction. Since the quaternized amine salt and the carboxyl acid in the hydroxy acid form ion pairs in a 1:1 molar ratio, it is desirable to add the hydroxy acid and the amine in the solvent so that the carboxyl group in the hydroxy acid of the above-mentioned general formula (II) and the amine group in the amine of the above-mentioned general formula (III) become equivalent. Although the above-mentioned reaction is dependent on the solubility of the starting materials, namely, the hydroxy acid and the amine, in the solvent and the type of solvent used, it is normally performed for a short period of time at a temperature of about 10 to 40° C. After reacting, the product precipitates out in the solvent and the product can be separated from the reaction product by a separation means such as filtering or the like. The hydroxy acid amine salt of the above-mentioned general formula (I) of the present invention can be obtained in a high yield of almost 100% by this comparatively simple synthesis method. The solvent used in the above-mentioned synthesis reaction can be arbitrarily determined considering solubility of the starting materials and ease of separation from the product. As specific examples of the solvent, methanol, acetone, 2-propanol, toluene, hexane, ethanol, ethylmethylketone, butanol, pentanol, hexanol, and the like may be mentioned.

As specific examples of the hydroxy acid of the above-mentioned general formula (II) used as a starting material in this production method, for example, the saturated or unsaturated chain hydrocarbon groups, hetero atom-containing chain hydrocarbon groups, alicyclic hydrocarbon groups, aromatic hydrocarbon groups, heterocyclic groups, and hydroxy-substituted derivatives that are hydroxy-substituted derivatives of these groups and that have 1 or more hydroxyl groups disclosed as specific examples of R¹ in general formula (I) may be mentioned. Preferably, the hydroxy acid of the above-mentioned general formula (II) has a saturated or unsaturated chain hydrocarbon group of 1 to 12 carbons having 1 or more hydroxyl groups or an aromatic hydrocarbon group of 6 to 12 carbons having 1 or more hydroxyl groups as the R¹ group. More preferably, the above-mentioned hydroxy acid has hydrocarbon group of 1 to 8 carbons having 1 or more hydroxyl groups or an aromatic hydrocarbon group of 6 to 12 carbons having 1 or more hydroxyl groups, for example, a hydroxymethylene group (—CH(OH)—), a monohydroxyethylene group (—CH₂CH(OH)—), a 1,2-dihydroxyethylene group (—CH(OH)—CH(OH)—), a 1-hydroxy-1,2,3-triylpropane group (—CH(OH)-CH₂—CH₂—), a 2-hydroxy-1,2,3-triylpropane group (—CH2—CH(OH)-CH₂—), a hydroxyphenylene group (—C₆H₃(OH)—) group, or the like as R¹. As examples of the above-mentioned more preferable hydroxy acids, aliphatic hydroxy acids such as tartronic acid, malic acid, tartaric acid, citramalic acid, citric acid, isocitric acid, and the like; aromatic hydroxy acids such as 5-hydroxyisophthalic acid and the like; and similar hydroxy acids may be mentioned. Even more preferably, the hydroxy acid has a monohydroxyethylene group (—CH₂CH(OH)—) or a 1,2-dihydroxyethylene group (—CH(OH)—CH(OH)—) as the R¹ group. As examples of hydroxy acids having a monohydroxyethylene group (—CH₂CH(OH)—) or a 1,2-dihydroxyethylene group (—CH(OH)—CH(OH)—) as the R¹ group, malic acid and tartaric acid may be mentioned.

The amine of above-mentioned formula (III) that is used as a starting material of the amine salt of the present invention can be a primary, secondary, or tertiary amine. As examples of primary amines, primary amines having 1 group selected from saturated or unsaturated chain hydrocarbon groups, alicylic hydrocarbon groups, aromatic hydrocarbon groups, and heterocyclic groups disclosed as specific examples of R², R³, and R⁴ in general formula (I) as an N-substituent, for example, methylamine, ethylamine, propylamine, isopropylamine, butylamine, sec-butylamine, tert-butylamine, pentylamine, hexylamine, 2-ethylhexylamine, heptylamine, octylamine, nonylamine, decylamine, undecylamine, dodecylamine, ethylenediamine, hexamethylenediamine, methoxyamine, cyclopropylamine, cyclobutylamine, cyclopentylamine, cyclohexylamine, cycloheptylamine, cyclooctylamine, amantadine, aniline, benzylamine, phenylethylamine, 2-,3- or 4-aminotoluene, 2,3-, 2,4-, 2,5-, 2,6-, 3,4-, or 3,5-dimethylaniline, 2,4,5- or 2,4,6-trimethylaniline, 2,3,4,5-, 2,3,5,6- or 2,3,4,6-tetramethylaniline, 2-, 3-, or 4-methoxyaniline, 2-, 3-, or 4-ethoxyaniline, 2- or 4-methoxy-3-methylaniline, 2- or 3-methoxy-4-methylaniline, 2-, 3-, or 4-methoxy-5-methylaniline, 2- or 3-methoxy-6-methylaniline, 3- or 4-methoxy-2-methylaniline, 2- or 4-methoxy-3-ethylaniline, 2- or 3-methoxy-4-ethylaniline, 2-, 3-, or 4-methoxy-5-ethylaniline, 2-, 3-, or 4-methoxy-6-ethylaniline, 3- or 4-methoxy-2-ethylaniline, 2-methoxy-3,4,5-trimethylaniline, 3-methoxy-2,4,5-trimethylaniline, 4-methoxy-2,3,5-trimethylaniline, dopamine, and the like may be mentioned.

As specific examples of secondary amines, secondary amines having two groups selected from saturated or unsaturated chain hydrocarbon groups, alicylic hydrocarbon groups, aromatic hydrocarbon groups, and heterocyclic groups disclosed as specific examples of R², R³, and R⁴ in general formula (I) as N-substituents, for example, dimethylamine, diethylamine, dipropylamine, diallylamine, N,N′-dimethylethylenediamine, N-methylcyclohexylamine, N-ethylcyclohexylamine, N,N-dicyclohexylamine, N-methylaniline, N-cyclohexylaniline, N-allylaniline, diphenylamine, 4,4′-dimethyldiphenylamine, N-methyl-N-benzylamine, N-ethyl-N-benzylamine, N-cyclohexyl-N-benzylamine, N-cyclohexylaniline, and the like may be mentioned.

As specific examples of tertiary amines, tertiary amines having three groups selected from saturated or unsaturated chain hydrocarbon groups, alicylic hydrocarbon groups, aromatic hydrocarbon groups, and heterocyclic groups disclosed as specific examples of R², R³, and R⁴ in general formula (I) as N-substituents, for example, trimethylamine, triethylamine, tetramethylethylenediamine, N,N-diisopropylethylamine, N,N-dimethylcyclohexylamine, N-methyl-dicyclohexylamine, tricyclohexylamine, N,N-dimethylaniline, N,N-dimethyl-1-naphthylamine, 1,8-bis(dimethylamino)-naphthalene, 4-(dimethylamino)pyridine, N,N-dimethylbenzylamine, N,N-diethylbenzylamine, N-benzyl-N-ethylaniline, N,N-dibenzylmethylaniline, tribenzylamine, and the like may be mentioned.

Preferably, the amine of the above-mentioned general formula (III) is selected from amines having groups selected from saturated or unsaturated chain hydrocarbons of 4 to 6 carbons, saturated or unsaturated alicyclic hydrocarbons of 6 carbons, and aromatic hydrocarbons of 6 to 8 carbons as R², R³, and R⁴. More preferably, the amine of the above-mentioned general formula (III) is a mono- or di-primary amine having 1 or 2 amine groups per 1 molecule. As examples of preferable amines, tert-butylamine, hexamethylenediamine, cyclohexylamine, and phenylethylamine may be mentioned.

The rubber composition of the third aspect of the present invention contains the hydroxy acid amine salt represented by the above-mentioned general formula (I) in the rubber component. The rubber component of the rubber composition can be constituted from natural rubber (NR); diene-based synthetic rubbers such as butadiene rubber (BR), styrene-butadiene copolymer rubber (SBR), polyisoprene rubber (IR), acrylonitrile-butadiene copolymer rubber (NBR), chloroprene rubber (CR), ethylene-propylene-diene copolymer rubber (EPDM), styrene-isoprene copolymer rubber, styrene-isoprene-butadiene copolymer rubber, isoprene-butadiene copolymer rubber; and the like. Also, one or more types of the above-mentioned natural rubber and the diene-based synthetic rubber may be combined as the rubber component of the rubber composition. When natural rubber and the diene-based synthetic rubber are combined and used, the blending ratio of the natural rubber and diene-based synthetic rubber is arbitrary.

The rubber composition of the present invention preferably contains 0.5 to 10 parts by weight and more preferably 0.5 to 5 parts by weight of the hydroxy acid amine salt represented by the above-mentioned general formula (I) per 100 parts by weight of the rubber component.

In addition to the above-mentioned hydroxy acid amine salt, various compounding agents and additives normally added to rubber compositions such as reinforcing fillers; vulcanization or cross-linking agents; vulcanization or cross-linking accelerators; vulcanization or cross-linking promoters such as stearic acid, zinc oxide, and the like; various oils, anti-oxidants; softeners; plasticizers; and the like can be added to the rubber composition of the present invention by normal blending methods in normally used amounts according to the various uses thereof

As specific examples of reinforcing fillers, carbon black, silica, calcium carbonate, talc, clay, and the like may be mentioned. Although the amount of the reinforcing filler blended depends on the use of the rubber composition, it is normally 20 to 100 parts per weight per 100 parts of the rubber component in order to increase the mechanical strength of the rubber. When the rubber composition of the present invention is used to form a rubber member of a pneumatic tire, as examples of carbon black that can be used in the rubber composition of the present invention, carbon blacks of the grades SAF-HS, SAF, ISAF-HS, ISAF, ISAF-LS, IISAF-HS, HAF-HS, HAF, and HAF-LS may be mentioned. When silica is added as a reinforcing filler to the rubber composition of the present invention, although the silica can be blended in an arbitrary proportion into the reinforcing filler, the total amount of the reinforcing filler is preferably 65 to 100 parts by weight from the point of securing reinforcement. When silica is added as a reinforcing filler, 2 to 15 percent by weight of a silane coupling agent is added with respect to the total weight of the silica in order to increase dispersibility of the silica in the rubber component.

As the mixing method used when blending the additives, normal methods can be used, and normally the compounding agents can be mixed in lump form, pellet form, or powder form using a suitable mixer, for example, a kneader, an internal mixer, a Banbury mixer, a roll, or the like. After the rubber composition is prepared by mixing the various compounding agents, a tire tread, for example, can be formed by a normal pressure molding method.

### EXAMPLES

The present invention is further explained in detail, referring to the following examples and comparative examples. However, it goes without saying that the technical scope of the present invention is not limited by these examples.

### Analysis Techniques

The hydroxy acid amine salt (malic acid cyclohexylamine salt) used in the below-mentioned Example 1, the hydroxy acid amine salt (tartaric acid cyclohexylamine salt) used in the below-mentioned Example 2, and the amine salt for comparison (succinic acid cyclohexylamine salt) used in Comparative Example 2 were synthesized by the below-mentioned synthesis methods, and the chemical structures thereof were identified as a result of performing nuclear magnetic resonance spectroscopy (¹H-NMR and ¹³C-NMR) and an elemental analysis technique by the below-mentioned procedures.

### (1) Nuclear Magnetic Resonance Spectroscopy (¹H-NMR and ¹³C-NMR)

Nuclear magnetic resonance spectroscopy (¹H-NMR and ¹³C-NMR) was performed using a nuclear magnetic resonance spectrometer AV400M (400 MHz) made by Bruker and using heavy dimethyl sulfoxide (heavy DMSO) as the solvent.

### (2) Elemental Analysis Technique

Elemental analysis was performed using a fully automatic elemental analyzer 240011 made by PerkinElmer. This elemental analyzer determined the quantity of each of the elements carbon, hydrogen, nitrogen, and oxygen using frontal chromatography by completely burning the sample at a high temperature of 1,800° C. or more in oxygen. The proportion of each of the elements carbon, hydrogen, and nitrogen are represented as percent by weight.

### Synthesis of Hydroxy Acid Amine Salts

### 1. Synthesis of Malic Acid Cyclohexylamine Salt

150 mL of acetone was added to a 1 liter round-bottom flask with a cork. Next, 60 g (0.447 mol) of malic acid and 88.7 g (0.894 mol) of cyclohexylamine was added thereto, and a precipitate formed after reacting for 5 minutes at room temperature. By filtering the precipitate, washing the precipitate remaining on the filter paper twice with acetone, and drying under reduced pressure, 147.0 g (yield: 99%) of a powdered white product was obtained. This product had a melting point of 149.9° C. This product was analyzed by the above-mentioned nuclear magnetic resonance spectroscopy (¹H-NMR and ¹³C-NMR) and elemental analysis technique, and was identified as the malic acid cyclohexylamine salt represented by the following structural formula.

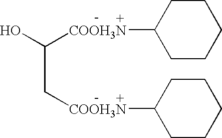

¹H- and ¹³C-NMR Measurement Results:

¹H-NMR spectrum (400 MHz, heavy DMSO) chemical shift δ (ppm): 1.0-1.3, 1.6, 1.7, 1.8, 2.3, 2.5, 2.7, 3.8

¹³C-NMR spectrum (400 MHz, heavy DMSO) chemical shift δ (ppm): 24.1, 24.9, 33.3, 42.2, 49.6, 175.0, 176.2

Elemental Analysis Results:

Measured values: C 57.97; H 10.00; N 8.39

Calculated values: C 57.81; H 9.70; N 8.43

### 2. Synthesis of Tartaric Acid Cyclohexylamine Salt

300 mL of acetone was added to a 1 liter round-bottom flask with a cork. Next, 42 g (0.28 mol) of tartaric acid and 64 mL (0.56 mol) of cyclohexylamine were added thereto, and a precipate formed after reacting for 10 minutes at room temperature. By filtering the precipitate, washing the precipitate remaining on the filter paper twice with acetone, and drying under reduced pressure, 95.6 g (yield: 98%) of a white powder product was obtained. This product had a melting point of 157.71° C. This product was analyzed by the above-mentioned nuclear magnetic resonance spectroscopy (¹H-NMR and ¹³C-NMR) and elemental analysis technique, and was identified as the succinic acid cyclohexylamine salt represented by the following structural formula.

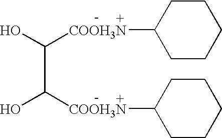

¹H- and ¹³C-NMR Measurement Results:

¹H-NMR spectrum (400 MHz, heavy DMSO) chemical shift δ (ppm): 1.0-1.2, 1.6, 1.7, 1.9, 2.9, 3.8

¹³C-NMR spectrum (400 MHz, heavy DMSO) chemical shift δ (ppm): 23.7, 24.5, 30.4, 49.2, 71.2, 174.3

Elemental Analysis Results:

Calculated values: C 55.15; H 9.26; N 8.04; O 27.55

Measured values: C 49.61; H 8.27; N 6.09; O 34.49

### 3. Synthesis of Succinic Acid Cyclohexylamine Salt (Amine Salt for use in Comparison)

150 mL of acetone was added to a 1 liter round flask with a cork. Next, 60 g (0.508 mol) of succinic acid and 100.7 g (1.016 mol) of cyclohexylamine were added thereto, and a precipate formed after reacting for 5 minutes at room temperature. By filtering the precipitate, washing the precipitate remaining on the filter paper twice with acetone, and drying under reduced pressure, 159.1 g (yield: 99%) of a powdered white product was obtained. This product had a melting point of 199.3° C. This product was analyzed by the above-mentioned nuclear magnetic resonance spectroscopy (¹H-NMR and ¹³C-NMR) and elemental analysis technique, and was identified as the succinic acid cyclohexylamine salt represented by the following structural formula.

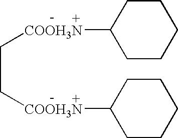

¹H- and ¹³C-NMR Measurement Results:

¹H-NMR spectrum (400 MHz, heavy DMSO) chemical shift δ (ppm): 1.0-1.3, 1.6, 1.8, 2.0, 2.4, 2.9

¹³C-NMR spectrum (400 MHz, heavy DMSO) chemical shift δ (ppm): 24.1, 24.6, 31.0, 33.9, 49.9, 180.3

### Elemental Analysis Results:

Calculated values: C 60.73; H 10.19; N 8.85

Measured values: C 61.06; H 10.53; N 9.29

### Preparation of Rubber Compositions of Comparative Examples 1 and 2 and Examples 1 and 2

In accordance with the formulations in Table 1 below, the compounding agents other than sulfur and the vulcanization accelerator were mixed using a 1.7 liter internal Banbury mixer for 5 minutes, and after discharging such from the mixer at 150° C., the sulfur and the vulcanization accelerator were mixed therein for 3 minutes using a roll to obtain the unvulcanized rubber compositions of Comparative Examples 1 and 2 and Examples 1 and 2. The malic acid cyclohexylamine salt synthesized as above was blended into the rubber composition of Example 1 as the amine salt, the tartaric acid cyclohexylamine salt synthesized as above was blended into the rubber composition of Example 2 as the amine salt, and the succinic acid cyclohexylamine salt synthesized as above was blended into the rubber composition of Comparative Example 1 as the amine salt.

### Test Methods

### (1) Vulcanization Speed

With respect to each of the unvulcanized rubber compositions of Comparative Examples 1 and 2 and Examples 1 and 2 as prepared above, in accordance with “vulcanization test with an oscillating vulcanization tester” of JIS K6300, the change in viscosity over time at a temperature of 160° C. and an amplitude angle of 1 degree was recorded as a torque-time curve (vulcanization curve) with torque (load) as the vertical axis and vulcanization time (minutes) as the horizontal axis, and this curve was analyzed to determine the time (T95) (minutes) until 95% of the maximum torque value was reached. The smaller the numerical value of T95, the faster the vulcanization speed.

### (2) Mooney Scorch Time

With respect to each of the unvulcanized rubber compositions of Comparative Examples 1 and 2 and Examples 1 and 2, the Mooney viscosity was continuously measured in accordance with JIS K6300 using an L-type rotor under the conditions of a preheating time of 1 minute and a test temperature of 125° C. The Mooney scorch time (ML 5UP) (minutes) until the value for Mooney viscosity increased 5 points from the minimum value Vm from the start of residual heat was determined. The tests results are as shown in Table 1. The Mooney scorch time (ML 5UP) is an indicator of scorching (rubber scorching), with longer times being better. “>45” for “ML 5UP” means that that the measurement of Mooney viscosity was stopped at 45 minutes.

### (3) Tensility Test

Each unvulcanized rubber composition of Comparative Examples 1 and 2 and Examples 1 and 2 was press vulcanized at 160° C. for 30 minutes to obtain a vulcanized rubber sheet having a length of 15 cm, a width of 15 cm, and a thickness of 2 mm A JIS No. 3 dumbbell-shaped test piece was punched from this vulcanized rubber sheet. Next, with respect to the test pieces of Comparative Examples 1 and 2 and Examples 1 and 2, in accordance with JIS K6251, the modulus (M100) (MPa) at 100% elongation (hereinafter referred to as “M100 before aging”, the break stress (T_{B}) (MPa) (hereinafter referred to as “T_{B} before aging”), and the elongation at break (E_{B}) (%) (hereinafter referred to as “E_{B} before aging”) were determined. (4) Aging Test

JIS No. 3 dumbbell-shaped test pieces were prepared from each of the unvulcanized rubber compositions of Comparative Examples 1 and 2 and Examples 1 and 2 in accordance with the same procedure disclosed in the tensility test above. The vulcanized rubber test pieces obtained were aged by heating for 96 hours in a temperature-controlled bath of 80° C. Next, the modulus (M100) (MPa) at 100% elongation, the break stress (T_{B}) (MPa), and the elongation at break (E_{B}) (%) after aging were determined. Next, in accordance with JIS K6257, the rates of change (%) in the values of M100, T_{B}, and E_{B} after aging with respect to M100, T_{B}, and E_{B} before aging were calculated for each of the vulcanized rubber test pieces of Comparative Examples 1 and 2 and Examples 1 and 2.

The test results are shown in Table 1 below. In Table 1, the values of T95, M100 before aging, T_{B} before aging, and E_{B} before aging of Comparative Example 2 and

Examples 1 and 2 are represented by indices as relative values when the values of T95, M100 before again, T_{B} before aging, and E_{B} before aging of Comparative Example 1 are 100 each

TABLE 1

| | Comparative | Comparative | | |
|---|---|---|---|---|
| | Example 1 | Example 2 | Example 1 | Example 2 |
| Formulation | | | | |
| NR⁽¹⁾ | 100.00 | 100.00 | 100.00 | 100.00 |
| Carbon black⁽²⁾ | 50.00 | 50.00 | 50.00 | 50.00 |
| Zinc oxide⁽³⁾ | 3.00 | 3.00 | 3.00 | 3.00 |
| Stearic acid⁽⁴⁾ | 1.00 | 1.00 | 1.00 | 1.00 |
| Anti-oxidant⁽⁵⁾ | 1.50 | 1.50 | 1.50 | 1.50 |
| Sulfur⁽⁶⁾ | 1.20 | 1.20 | 1.20 | 1.20 |
| Vulcanization | 1.20 | 1.20 | 1.20 | 1.20 |
| accelerator⁽⁷⁾ | | | | |
| Amine salt | — | 1.00 | 1.00 | 1.00 |
| Test Results | | | | |
| T95 | 100 | 83.5 | 58.6 | 75.9 |
| ML 5UP | 42.8 | >45 | >45 | 44.3 |
| M100 before aging | 100 | 103.3 | 116.7 | 113.3 |
| T_{B} before aging | 100 | 101.3 | 99.3 | 100.3 |
| E_{B} before aging | 100 | 98.2 | 93.1 | 96.1 |
| Rate of change (%) of | 139 | 139 | 123 | 92.8 |
| M100 | | | | |
| Rate of change (%) | 88 | 91 | 94 | 105.7 |
| of T_{B} | | | | |
| Rate of change (%) | 75 | 77 | 81 | 104 |
| of E_{B} | | | | |

Footnotes of Table 1:
⁽¹⁾Natural rubber (TSR20)
⁽²⁾NITERON #20018 made by Nippon Steel Chemical Carbon Co., Ltd.
⁽³⁾Zinc Oxide #3 made by Seido Chemical Industry Co., Ltd.
⁽⁴⁾Industrial stearic acid made by Chiba Fatty Acid Co., Ltd.
⁽⁵⁾6PPD made by Flexsys
⁽⁶⁾Powdered sulfur made by Karuizawa Seirensho
⁽⁷⁾Sanceler-CM-G made by Sanshin Chemical Industry Co., Ltd.

From the results shown in Table 1, it is understood that rubber compositions containing the hydroxy acid amine salt of the present invention show superior vulcanization characteristics and viscoelastic characteristics.

1. A hydroxy acid amine salt represented by the following general formula (I): 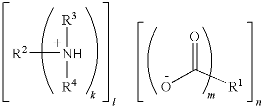
(wherein R¹ represents a saturated or unsaturated organic group of 1 to 12 carbons having 1 or more hydroxyl groups;
R², R³, and R⁴ each independently represent a hydrogen atom, a saturated or unsaturated chain hydrocarbon group of 1 to 12 carbons, a saturated or unsaturated alicyclic hydrocarbon group of 3 to 12 carbons, an aromatic hydrocarbon group of 6 to 18 carbons, or a heterocyclic group of 5 to 18 ring atoms and 2 or more carbons, or at least two of R², R³, and R⁴ join together with nitrogen atoms to which they are bonded to form a heterocyclic group of 5 to 18 ring atoms and 2 or more carbons;
and k, l, and n are each integers of 1 or more, and m is an integer of 2 or more, with a proviso that k, l, m, and n satisfy a relational expression k×l=m×n=2 or more).

2. The hydroxy acid amine salt according to claim 1, wherein the number of carbons of R¹ in general formula (I) is 2.

3. A method for producing the hydroxy acid amine salt according to claim 1, the method comprising reacting n moles of a hydroxy acid represented by the following general formula (II):
R¹—(COOH)ₘ (II)
(wherein R¹ represents a saturated or unsaturated organic group of 1 to 12 carbons having 1 or more hydroxyl groups, and m is an integer of 2 or more)
with 1 mole of an amine represented by the following general formula (III):
R²—(NR³R⁴)_{K} (III)
(wherein R², R³, and R⁴ each independently represent a hydrogen atom, a saturated or unsaturated chain hydrocarbon group of 1 to 12 carbons, a saturated or unsaturated alicyclic hydrocarbon group of 3 to 12 carbons, an aromatic hydrocarbon group of 6 to 18 carbons, or a heterocyclic group of 5 to 18 ring atoms and 2 or more carbons, or at least two of R², R³, and R⁴ join together with nitrogen atoms to which they are bonded to form a heterocyclic group of 5 to 18 ring atoms and 2 or more carbons, and k is an integer of 1 or more).

4. A rubber composition comprising 0.5 to 10 parts by weight, per 100 parts by weight of a rubber component, of the hydroxy acid amine salt according to claim 1.

5. The hydroxy acid amine salt according to claim 1, wherein R¹ has one or two aromatic groups.

6. The hydroxy acid amine salt according to claim 1, wherein R¹ has from 1 to 8 carbon atoms.

7. The hydroxy acid amine salt according to claim 1, wherein at least one of R¹, R², R³, or R⁴ comprises a straight hydrocarbon chain.

8. The hydroxy acid amine salt according to claim 1, wherein at least one of R¹, R², R³, or R⁴ comprises a branched carbon chain.

9. The hydroxy acid amine salt according to claim 1, wherein R¹ has from 2 to 4 carbons.

10. The hydroxy acid amine salt according to claim 1, wherein at least one of R², R³, and R⁴ includes an aromatic group and a total of 6 to 8 carbons.

11. The hydroxy acid amine salt according to claim 1, wherein at least one of R², R³, and R⁴ includes an alicyclic group of 6 carbons.

12. The hydroxy acid amine salt according to claim 1, wherein k=1, l=2, m=2, and n=1.

13. The hydroxy acid amine salt according to claim 1, wherein k=1, l=3, m=3, and n=1.

14. The method for producing the hydroxy acid amine salt according to claim 3, wherein reacting the hydroxy acid moiety with the amine moiety comprises reacting the hydroxy acid and the amine with a solvent.

15. The method for producing the hydroxy acid amine salt according to claim 16, further comprising separating the solvent and a product produced by the reaction using a filter.

16. The method for producing the hydroxy acid amine salt according to claim 3, wherein reacting the hydroxy acid with the amine comprises reacting the hydroxy acid and the amine without a solvent.

17. The rubber composition according to claim 4, wherein the hydroxyl acid amine salt is present at 0.5 to 5 parts by weight per 100 parts by weight of a rubber component.

18. The rubber composition according to claim 4, further comprising 65 to 100 parts per weight of a silica reinforcing filler per 100 parts of the rubber composition and 2 to 15 percent by weight of a silane coupling agent.
